(19) United States
(12) Patent Application Publication
(10) Pub. No.: US 2007/0037266 A1
(43) Pub. Date: Feb. 15, 2007

(54) Process for producing erythritol

(76) Inventors: Thomas Sasman, Omaha, NE (US); William Alan Head, Omaha, NE (US); Corey A. Cameron, Omaha, NE (US)

Correspondence Address: CARGILL, INCORPORATED, LAW/24, 15407 MCGINTY ROAD WEST, WAYZATA, MN, 55391, US

(21) Appl. No.: 11/472,116

(22) Filed: Jun. 21, 2006

Related U.S. Application Data
(60) Provisional application No. 60/695,581, filed on Jun. 30, 2005.

(51) Int. Cl. C12P 7/18

(52) U.S. Cl. 435/158

(57) ABSTRACT

The disclosure relates to a process for producing erythritol that includes using an aqueous carbohydrate source that meets the requirements of 7 C.F.R. 205.102 (2002), and at least one other component that meets the requirements of 7 C.F.R. 205.605 (2002), to produce a media, and fermenting the media in the presence of a fermentation microorganism, in a fermenter that has been sterilized by indirect heating, or direct heating followed by a flush that removes chemical residue. The erythritol produced by the process may be referred to, or certified, as an organic erythritol. The disclosure also relates to a process for producing an erythritol-containing broth.

### RELATED APPLICATIONS

The present application claims the benefit of U.S. Provisional Application No. 60/695,581, filed Jun. 30, 2005, which is hereby incorporated by reference in its entirety.

### FIELD OF THE DISCLOSURE

The present disclosure is directed to a process for producing erythritol. The process utilized allows the erythritol that is produced to be referred to as organic erythritol.

### BACKGROUND OF THE DISCLOSURE

Erythritol is a known substance that is useful as a sweetener, and as an intermediate for the production of pharmaceuticals and industrial chemicals. Erythritol is a four carbon sugar alcohol, naturally occurring in a wide range of microorganisms, that is used as a low calorie sugar replacement in the food and pharmaceutical industry.

Typically, erythritol is commercially produced by fermentation of simple sugar. Many microorganisms are known to produce erythritol from simple sugar. For example, there may be used a strain of the yeast Moniliella Pollinis, Pichia, Candida, Torulopsis, Trigonopsis, Moniliella, Auriobasidium, Delbaryomyces, Aspergillus, Eurotium, Fennellia, and Leuconostoc. Industrial fermentation of erythritol is carried out in large scale fermenters. Medium, containing simple sugars as the carbon source, nitrogen, phosphate, and other minerals, is sterilized prior to inoculation with the strain of microorganism. The fermentation environment is carefully controlled to allow cell growth and erythritol production. As the fermentation is completed, the broth is heated for a period of time to kill the microorganisms and then filtered to remove the biomass from the erythritol rich process stream. The erythritol stream is purified by a series of steps that may include chromatic separation, ion exchange, filtration, and crystallization. Purified erythritol crystals are washed in a centrifuge to achieve 99.5% purity, dried in a drier to achieve at least 99.85% dry matter, sifted to properly size the finished product, and packaged for the market.

Exemplary processes for producing erythritol are found in the following U.S. patents:

U.S. Pat. No. 4,923,812 (EP 0 327 342 B1) is a process for continuously producing erythritol by cultivating erythritol-producing microorganisms under aerobic conditions. The process comprises the steps of: maintaining the concentration of dissolved oxygen in a culture broth in a fermentation tank at not less than 0.2 ppm; separating a part of the culture broth into a concentrated liquid in which the concentration of cells is increased and a clarified liquid is produced by a cell separator; returning the concentrated liquid to the fermentation tank; controlling an amount of the clarified liquid to be extracted outside an erythritol-producing system and an amount of the culture broth and/or the concentrated liquid to be extracted outside the producing system such that the concentration of the cells in the culture broth in the fermentation tank may be kept in a range from 40 to 200 g/l when calculated as a weight of dried cells; and recovering erythritol from the clarified liquid. The clarified liquid containing erythritol may be separated from the culture broth by a cell separator arranged inside the fermentation tank, and the extracting amount of the erythritol-containing clarified liquid separated by the cell separator and an amount of the culture broth to be extracted may be controlled such that the concentration of the cells in the culture broth may be kept at 40 to 200 g/l when calculated as a weight of dried cells.

U.S. Pat. No. 5,902,739 (EP 0 845 538 A2) is a method of producing erythritol by cultivating a yeast strain capable of producing erythritol from fermentable carbohydrate in a culture medium containing the carbohydrate as a main carbon source to recover erythritol from the culture, erythritol can be produced efficiently in high yield by using ammonium sulfate as a main nitrogen source in the culture medium.

U.S. Pat. No. 5,981,241 (EP 0 940 471 A1) is a method of producing erythritol, in which a microorganism having an ability of producing erythritol is cultivated for generation in a medium containing preferably 5 ppm or more of calcium, and erythritol is collected from the culture, thus producing erythritol efficiently.

U.S. Pat. No. 4,906,569 (EP 327 016 B1) is a process for readily isolating and recovering highly pure erythritol at a high crystallization yield from an erythritol-containing culture medium, which contains erythritol together with various impurities such as salts, coloring materials and polysaccharides, through chromatographic separation with the use of a strongly acidic cation exchange resin. The process can be continuously operated, since the lowered separation capability of said cation exchange resin can be readily restored by treating the same with a warm alkali solution.

U.S. Pat. No. 6,030,820 (EP 0 908 523 A3) is a process for producing a high-purity erythritol crystal comprising a crystallization step of subjecting an erythritol-containing aqueous solution as a raw solution to crystallization, wherein an erythritol concentration of said erythritol-containing aqueous solution is adjusted to 30 to 60% by weight at the beginning of the crystallization step; said erythritol-containing aqueous solution is cooled at a cooling rate of not more than 20° C./hour; a seed crystal of erythritol is added to said erythritol-containing aqueous solution in the course of the cooling, and the solution is cooled to not more than 20° C.

There is a desire to provide a process for producing erythritol, that will allow the resultant erythritol product to be referred to as an organically produced erythritol.

### SUMMARY OF THE DISCLOSURE

The present disclosure relates to any process for producing erythritol wherein the aqueous carbohydrate source of the media intended for fermentation comprises a carbohydrate source that meets the requirements of 7 C.F.R. 205.102 (2002), and wherein at least one other component of the media meets the requirements of 7 C.F.R. 205.605 (2002), and wherein the media is fermented in the presence of a fermentation microorganism, in a fermenter vessel (a fermenter) that has been sterilized by indirect heating, or by direct heating followed by a flush that removes chemical residue, prior to introduction of the media. All reference herein to 7 C.F.R. Part 205 relates to the Regulations effective in the year 2002.

### DETAILED DESCRIPTION OF THE DISCLOSURE

The present disclosure relates to any process for producing erythritol wherein the aqueous carbohydrate source of the media intended for fermentation comprises a carbohydrate source that meets the requirements of 7 C.F.R. 205.102 (2002), and wherein at least one other component of the media meets the requirements of 7 C.F.R. 205.605 (2002), and wherein the media is fermented in the presence of a fermentation microorganism, in a fermenter vessel (a fermenter) that has been sterilized by indirect heating, or by direct heating followed by a flush that removes chemical residue, prior to introduction of the media.

More particularly, the process for producing erythritol is described in detail, as follows.

As indicated above, the media requires the use of an aqueous carbohydrate source that meets the requirements of 7 C.F.R. 205.102 (2002). The aqueous carbohydrate source may comprise any amount of carbohydrate that allows production of a media. In one embodiment, the aqueous carbohydrate source comprises carbohydrate in an amount of about 200 to about 500 g/l. Suitable for use as the carbohydrate source are, but not limited to, sugar, and starch based dextrose obtained from starchy products such as corn, wheat, potato, or tapioca. The media also requires at least one other component that meets the requirements of 7 C.F.R. 205.605 (2002). These include, but are not limited to, minerals, a nitrogen source such as nutritive yeast, corn steep products, potassium phosphate, calcium phosphate, or the like, utilized as nutrients or processing aids, and ammonium carbonate used as a leavening agent. Antifoam additives are added to minimize foam formation. All the nutrients to be added to the fermenters are sterilized with indirect steam at a temperature of about 250° F. (121° C.) for 15 minutes, as the fermenters are filled. Subsequently, the nutrients are cooled to about 95° F. (35° C.).

The fermenters are sterilized prior to introducing media. The fermenters are sterilized by either indirect heating, or by direct heating followed by a flush that removes chemical residue, after sterilization, with sterilized water.

The sterilized fermenters are filled with sterilized media, and inoculated with a fermentation microorganism such as, in this instance, Moniliella Pollinis Organism strain No. S1477. Air is introduced to the fermenter. The pH of the broth is maintained above 3.5 by adding caustic. The continuous fermentation typically occurs over a period of about 120 hours, under agitation at about 175 rpm (revolutions per minute), and at a temperature of about 95° F. (35° C.), thereby causing conversion of the carbohydrate, such as sucrose or dextrose, to erythritol.

At the conclusion of fermentation, the contents of the fermenter are heated indirectly at a temperature of about 158° F.(70° C.) for a period of about 1 hour, to kill the M. Pollinis strain No. S1477.

Many microorganisms are known to produce erythritol from simple sugar. For example, there may be used a strain of the yeast Moniliella Pollinis, Pichia, Candida, Torulopsis, Trigonopsis, Moniliella, Auriobasidium, Delbaryomyces, Aspergillus, Eurotium, Fennellia, and Leuconostoc.

The heated broth is fed to a cell separation system to remove the biomass (the dead yeast cells) from the erythritol product stream. The liquid in the broth permeates a ceramic filter cell separation system, and the biomass-free permeate contains the erythritol product. The permeate containing the erythritol is maintained at a temperature of about 160° F. (71° C.). Where the cell separation employs a membrane filtration, the membranes may be cleaned by washing with sodium hydroxide, followed by flushing with clean water.

The erythritol containing biomass-free process stream is then softened using a weak acid ion exchange resin. Any weak acid ion exchange resin may be used to remove hardness, such as calcium and magnesium. Preferred is the use of weak acid ion exchangers such as Purolite's C-104 resin, or Mitsubishi's WK-20 resin.

Thereafter, the softened process fluid stream is concentrated by evaporation prior to chromatographic separation.

The concentrated process fluid stream is then subjected to chromatographic separation to remove most of the impurities. Any separation resin may be used. Typical separation resins include, but are not limited to, Purolite's PRC-821 resin, or Mitsubishi's UBK 550 F resin.

The purified process steam is then demineralized anionically and cationically to remove salinity. This is achieved by displacing positive cations with hydrogen ion, and negative anions with hydroxyl ion. The residence time is 6 hours, and the temperature of the demineralization is about 122° F. (50° C.). Suitable cationic resins include, but are not limited to, Purolite's C-155S, and Mitsubishi's PK 212 F resins. Suitable anionic resins include, but are not limited to, Purolite's A103S, and Mitsubishi's WA30 resins.

To remove residual salinity in the process fluid, further demineralization is achieved by treatment using a mixed bed column containing a mixture of strong-base anion exchange resin and strong-acid cation exchange resin. Typical examples of mixed bed systems are a combination of Purolite's C-155S and A51MBS resins and a combination of Mitsubishi's PK 212 F and PA-308 resins.

Thereafter, the demineralized process fluid stream is heated and concentrated, prior to crystallization.

The process stream is crystallized at a temperature of about 160° F. (71° C.), followed by reducing the temperature to about 59° F. (15° C.).

The foregoing process results in the production of highly purified erythritol product. Moreover, the resulting erythritol product may be identified or certified as being an organic erythritol. This is allowed since the erythritol is produced in compliance with the requirements of 7 C.F.R. Part 205, of the U.S. Department of Agriculture's National Organic Program.

The disclosure has been described with reference to various specific and illustrative embodiments and techniques. However, one skilled in the art will recognize that many variations and modifications may be made while remaining within the spirit and scope of the disclosure.

What is claimed is:

1. A process for producing an erythritol-containing broth comprising:
(a) combining an aqueous carbohydrate source that meets the requirements of 7 C.F.R. 205.102 (2002), with at least one other component that meets the requirements of 7 C.F.R. 205.605 (2002), to produce a media, and
(b) fermenting the resultant media, in the presence of a fermentation microorganism, in a fermenter that, prior to introduction of the media, has been sterilized by means of indirect heating, or by direct heating followed by a flush that removes chemical residue.

2. The process according to claim 1 wherein the carbohydrate source is selected from the group consisting of sugar and starch based dextrose.

3. The process according to claim 1 wherein the media comprising the carbohydrate source and the at least one other component is sterilized prior to introduction into the fermenter.

4. The process according to claim 1 wherein the fermentation microorganism is selected from the group consisting of Moniliella Pollinis, Pichia, Candida, Torulopsis, Trigonopsis, Moniliella, Auriobasidium, Debaryomyces, Aspergillus, Eurotium, Fennellia, and Leuconostoc.

5. The process according to claim 4 wherein the fermentation microorganism is Moniliella Pollinis.

6. The process according to claim 1 wherein the aqueous carbohydrate source comprises carbohydrate in an amount of about 200 to about 500 g/l.

7. In a process for producing erythritol, the improvement comprising producing an erythritol-containing broth by a process comprising:
(a) combining an aqueous carbohydrate source that meets the requirements of 7 C.F.R. 205.102 (2002), with at least one other component that meets the requirements of 7 C.F.R. 205.605 (2002),to produce a media; and
(b) fermenting the resultant media, in the presence of a fermentation microorganism, in a fermenter that, prior to introduction of the media, has been sterilized by means of indirect heating, or by direct heating followed by a flush that removes chemical residue.

8. The process according to claim 7 wherein the carbohydrate source is selected from the group consisting of sugar and starch based dextrose.

9. The process according to claim 7 wherein the media comprising the carbohydrate source and the at least one other component is sterilized prior to introduction into the fermenter.

10. The process according to claim 7 wherein the fermentation microorganism is selected from the group consisting of Moniliella Pollinis, Pichia, Candida, Torulopsis, Trigonopsis, Moniliella, Auriobasidium, Debaryomyces, Aspergillus, Eurotium, Fennellia, and Leuconostoc.

11. The process according to claim 10 wherein the fermentation microorganism is Moniliella Pollinis.

12. The process according to claim 7 wherein the aqueous carbohydrate source comprises carbohydrate in an amount of about 200 to about 500 g/l.

13. A process for producing erythritol comprising:
(a) combining an aqueous carbohydrate source that meets the requirements of 7 C.F.R. 205.102 (2002), with at least one other component that meets the requirements of 7 C.F.R. 205.605 (2002),to produce a media,
(b) fermenting the resultant media, in the presence of a fermentation microorganism, in a fermenter that, prior to introduction of the media, has been sterilized by means of indirect heating, or by direct heating followed by a flush that removes chemical residue, thereby producing an erythritol-containing broth;
(c) indirectly heating the broth to kill fermentation microorganism;
(d) separating killed microorganism cells from the heated broth to provide a biomass—free erythritol product stream;
(e) maintaining the erythritol product stream at a temperature of about 160° F. (71° C.);
(f) contacting the erythritol product stream with a weak acid ion exchange resin to soften the erythritol product stream;
(g) concentrating the softened erythritol product stream by evaporation;
(h) subjecting the concentrated erythritol product stream to chromatographic separation;
(i) demineralizing the chromatographically separated erythritol product stream anionically, cationically, and then with a mixed anionic and cationic demineralization;
concentrating the demineralized erythritol product stream; and
(k) crystallizing the concentrated erythritol product stream to produce crystallized erythritol.

14. The process according to claim 13 wherein the carbohydrate source is selected from the group consisting of sugar and starch based dextrose.

15. The process according to claim 13 wherein the media comprising the carbohydrate source and the at least one other component is sterilized prior to introduction into the fermenter.

16. The process according to claim 13 wherein the fermentation microorganism is selected from the group consisting of Moniliella Pollinis, Pichia, Candida, Torulopsis, Trigonopsis, Moniliella, Auriobasidium, Debaryomyces, Aspergillus, Eurotium, Fennellia, and Leuconostoc.

17. The process according to claim 16 wherein the fermentation microorganism is Moniliella Pollinis.

18. The process according to claim 13 wherein the fermentation is achieved over a period of about 120 hours, under agitation at about 175 revolutions per minute, and at a temperature of about 95° F. (35° C.).

19. The process according to claim 13 wherein the broth is indirectly heated to a temperature of about 158° F. (70° C.) for a period of about 1 hour, to kill the fermentation microorganism.

20. The process according to claim 13 wherein the erythritol product stream is demineralized over a period of about 6 hours, at a temperature of about 122° F. (50° C.).

21. The process according to claim 13 wherein the erythritol product stream is crystallized at a temperature of about 160° F. (71° C.), followed by reducing the temperature to about 59° F. (15° C.).

22. The process according to claim 13 wherein the aqueous carbohydrate source comprises carbohydrate in an amount of about 200 to about 500 g/l.
